(19) United States
(12) Patent Application Publication
(10) Pub. No.: US 2026/0157668 A1
(43) Pub. Date: Jun. 11, 2026

(54) OPTICAL SENSING MODULE

(71) Applicants: ZEBRIGHT INTELLIGENT TECH CO., LTD., Taoyuan County (TW); BRIGHTON PRECISION ENGINEERING CO., LTD., Taoyuan County (TW)

(72) Inventor: FENG-JU CHUANG, Taoyuan County (TW)

(21) Appl. No.: 19/268,106

(22) Filed: Jul. 14, 2025

(30) Foreign Application Priority Data
Dec. 6, 2024 (TW) 113147441

(51) Int. Cl. A61B 5/1455; A61B 5/00; A61B 5/145

(52) U.S. Cl. CPC A61B 5/14552; A61B 5/004; A61B 5/14532; A61B 5/681

(57) ABSTRACT

This disclosure provides an optical sensing module, and comprises a carrier board, at least one movable device, at least one light emitting unit, and at least one photo detector. The light emitting unit generates a detection light, and projects the detection light onto a subject to generate a diffuse reflection light. The movable device is disposed on the carrier board, and the light emitting unit is disposed on the movable device. The movable device drives the light emitting unit to move relative to the carrier board, so as to project the detection light onto different regions of the subject. The photo detector respectively receives multiple diffuse reflection lights from multiple regions. The multiple diffuse reflection lights are further analyzed, and one of the diffuse reflection lights is selected for calculation to improve the accuracy of the physiological parameters detected by the light sensing module.

### CROSS-REFERENCE TO RELATED APPLICATIONS

This non-provisional application claims priority claim under 35 U.S.C. § 119(a) on Taiwan Patent Application No. 113147441 filed Dec. 6, 2024, the entire contents of which are incorporated herein by reference.

### TECHNICAL FIELD

This disclosure is an optical sensing module that is able to quickly and accurately measure the physiological parameters of a subject in a non-invasive manner.

### BACKGROUND

Driven by the increasing public awareness of health, a proliferation of wearable devices designed to monitor users' physiological parameters, such as smartwatches, smart bracelets, and smart necklaces, has emerged in the market. These devices are capable of measuring a range of physiological indicators, including blood pressure, blood oxygen saturation, heart rate, blood glucose levels, and/or body temperature. Furthermore, the wearable devices facilitate the storage of measured data, or the transmission of such data to a smartphone for prolonged health monitoring, thereby assisting medical professionals in assessing users' physiological conditions.

In the event of anomalous physiological parameters, these wearable devices or associated smartphones are capable of generating alerts, or sending notification to designated contacts, including healthcare providers, through network connectivity, thereby mitigating potential risks to the user.

Although wearable devices have the advantages of being lightweight and small in size, and are convenient for users to wear for long periods, the wearable devices currently on the market generally have the problem of inaccurate measurement of physiological parameters.

Specifically, wearable devices primarily project a detection light onto the user's (subject's) skin through a light-emitting unit, allowing the detection light to enter the lower layer of the skin and project onto blood vessels. Subsequently, a photo detector detects diffuse reflection light, which is reflected and scattered by the blood vessels. This process determines the extent of spectral absorption, reflection, and refraction, and in conjunction with algorithmic processing, enables the calculation of the user's physiological parameters.

However, when the detection light fails to project onto the main blood vessels in the lower layer of the skin, it may cause errors in the measured physiological parameters, thereby reducing the accuracy of judging the user's physical condition.

### SUMMARY

Thus, this invention provides an optical sensing module capable of projecting a detection light to varying regions on a subject, and subsequently selecting a more precise diffuse reflection light for computational analysis, thereby enhancing the accuracy of physiological parameters measured by the optical sensing module.

One object of this invention is to provide an optical sensing module, including a carrier board, a movable device, a light emitting unit and a photo detector, wherein the light emitting unit and/or the photo detector are disposed on the movable device. The movable device can be used to drive the light emitting unit and/or the photo detector to displace relative to the carrier board, so that the detection light generated by the light emitting unit can be projected to varying regions of the subject, and the varying regions of the subject are measured respectively, which is beneficial to improve the accuracy of the measured physiological parameters.

To achieve the foregoing objectives, this disclosure provides an optical sensing module, comprising: a carrier board, at least one light emitting unit, at least one movable device and at least one photo detector. The light emitting unit is configured to generate a detection light, and the detection light is configured to project onto a subject to generate a diffuse reflection light. The movable device is disposed on the carrier board, and includes a slide rail and a slider. The slider is displaceable along the slide rail, and the light emitting unit is disposed on the slider. The movable device is used to drive the light emitting unit to displace relative to the carrier board to project the detection light onto different positions of the subject. The photo detector is configured to receive the diffuse reflection light.

This disclosure provides another optical sensing module, comprising: a carrier board, at least one light emitting unit, at least one photo detector, and a first movable device. The light emitting unit is configured to generate a detection light, and the detection light is configured to project onto a subject to generate a diffuse reflection light. The photo detector is configured to receive the diffuse reflection light. A first movable device is disposed on the carrier board, and includes a first slide rail and a first slider. The first slider is able to displace along the first slide rail, and the photo detector is disposed on the first slider. The first slider is configured to drive the photo detector to displace relative to the carrier board, enabling the photo detector to receive the diffuse reflection light at varying positions.

The light sensing module described in this invention has the following advantages: by driving the light emitting unit and/or the photo detector to displace through the movable device, and measuring different positions of the subject, the physiological parameters of the subject can be measured and monitored quickly and accurately.

### BRIEF DESCRIPTION OF THE DRAWINGS

FIG. 1 is a schematic diagram of a wearable device with an optical sensing module according to an embodiment of the invention.

FIG. 2 is a structural diagram of an optical sensing module according to an embodiment of the invention.

FIG. 3 is a structural diagram of the optical sensing module according to another embodiment of the invention.

FIG. 4 is a structural diagram of the optical sensing module according to another embodiment of the invention.

FIG. 5 is a structural diagram of the optical sensing module according to another embodiment of the invention.

FIG. 6 is a structural diagram of the optical sensing module according to another embodiment of the invention.

FIG. 7 is a structural diagram of the optical sensing module according to another embodiment of the invention.

FIG. 8 is a structural diagram of the optical sensing module according to another embodiment of the invention.

FIG. 9 is a structural diagram of the optical sensing module according to another embodiment of the invention.

FIG. 10 is a structural diagram of the optical sensing module according to another embodiment of the invention.

FIG. 11 is a side view of the optical sensing module according to another embodiment of the invention.

FIG. 12 is a side view of the optical sensing module according to another embodiment of the invention.

FIG. 13 is a side view of the optical sensing module according to another embodiment of the invention.

FIG. 14 is a schematic diagram of the wearable device with the optical sensing module according to another embodiment of the invention.

FIG. 15 is a schematic diagram of the wearable device with the optical sensing module according to another embodiment of the invention.

### DETAILED DESCRIPTION

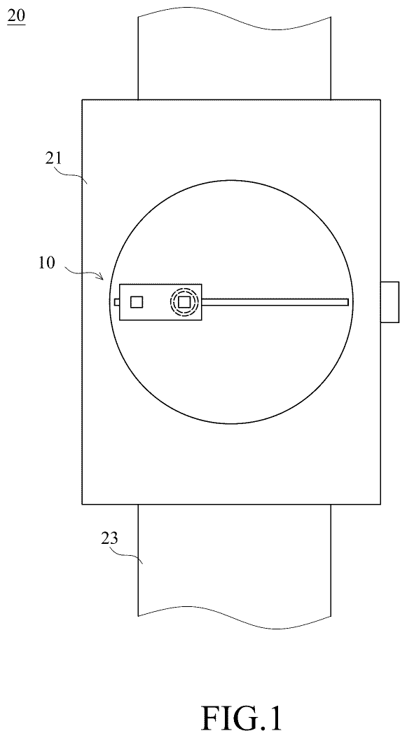

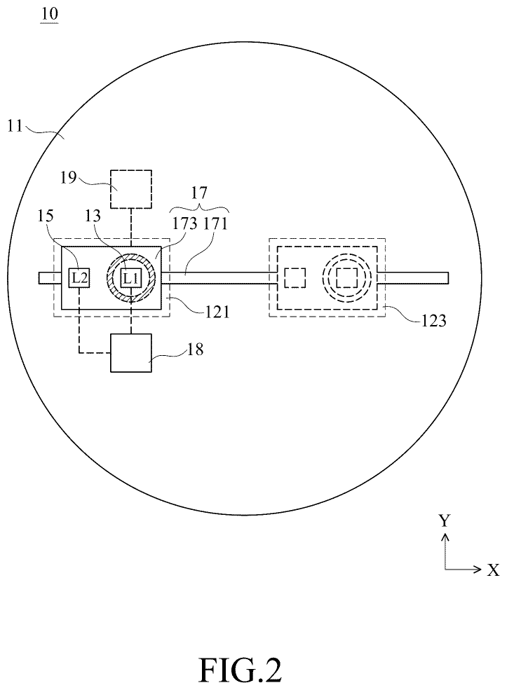

FIG. 1 is a schematic diagram of a wearable device with an optical sensing module according to an embodiment of the invention. FIG. 2 is a schematic diagram the optical sensing module according to an embodiment of the invention. The optical sensing module 10 of the invention can be disposed in a wearable device 20. For example, the wearable device 20 includes but is not limited to a smart watch, and the optical sensing module 10 can be disposed on the watch body 21 of the wearable device 20.

The optical sensing module 10 includes at least one carrier board 11, at least one light emitting unit 13, at least one photo detector 15, and a movable device 17. The light emitting unit 13 and/or the photo detector 15 are disposed on the carrier board 11 through the movable device 17, so that the light emitting unit 13 and/or the photo detector 15 can be displaced relative to the carrier board 11.

The carrier board 11 is used to carry the movable device 17, the light emitting unit 13 and the photo detector 15. For example, the carrier board 11 includes, but is not limited to, a circuit board, a glass substrate, an organic resin substrate or a wafer.

The movable device 17 is disposed on the carrier board 11. In one embodiment of the invention, the movable device 17 may include a slide rail 171 and a slider 173. The slider 173 is disposed on the slide rail 171 and can be displaced along the slide rail 171. The movable device 17 including the slide rail 171 and the slider 173 is only an embodiment of the invention and is not a limitation of the scope of the invention. For example, the movable device 17 may be a connecting rod or a robotic arm.

In one embodiment of the invention, as shown in FIG. 2, both the light emitting unit 13 and the photo detector 15 are disposed on the movable device 17, and the movable device 17 drives the light emitting unit 13 and the photo detector 15 to displace relative to the carrier board 11. For example, the light emitting unit 13 and the photo detector 15 are disposed on the same slider 173, wherein the relative position and distance between the light emitting unit 13 and the photo detector 15 are kept fixed, and the slider 173 drives the photo detector 15 and the light emitting unit 13 to displace along the slide rail 171. When the light emitting unit 13 and the photo detector 15 are displaced relative to the carrier board 11 along the slide rail 171, the distance between the two will remain constant, and general algorithms can be used to calculate the physiological parameters of the subject.

In practical applications, the light emitting unit 13 and the photo detector 15 can be located at a first position 121, wherein the light emitting unit 13 at the first position 121 projects a detection light L1 to a first region of the subject. In other embodiments, the subject may be animals, plants, or foods other than the human body, such as meat, eggs, vegetables, etc., and the quality of the food can be detected through the optical sensing module 10.

Part of the detection light L1 may be reflected or scattered by the surface of the first region of the subject, and part of the detection light may enter the interior of the subject and be absorbed, reflected or scattered by the internal tissues of the subject, such as being absorbed, reflected or scattered by blood vessels in the first region of the subject. The light reflected and scattered by the surface and internal tissues of the subject can be defined as a diffuse reflection light L2, and the diffuse reflection light L2 of the first region can be received by the photo detector 15.

In addition, the light emitting unit 13 and the photo detector 15 can be moved to a second position 123 by the slider 173 along the slide rail 171. The light emitting unit 13 located at the second position 123 projects the detection light L1 to the second region of the subject, and the photo detector 15 is used to receive the diffuse reflection light L2 of the second region.

The aforementioned first position 121 is different from the second position 123, and the first region of the subject is different from the second region. In other words, by the arrangement of the movable device 17, the light emitting unit 13 is able to project the detection light L1 to different regions of the subject, and then the photo detector 15 is able to sense the diffuse reflection light L2 of different regions of the subject.

As described in the prior art, when the detection light fails to project onto the correct region of the subject, such as failing to project onto the main blood vessels in the lower layer of the skin, it may cause inaccurate measurement of physiological parameters.

Compared to the prior art, the light emitting unit 13 and/or the photo detector 15 of the invention are connected to the carrier board 11 through the movable device 17, and the detection light L1 generated by the light emitting unit 13 can project onto different regions of the subject. Specifically, the light emitting unit 13 can be displaced relative to the carrier board 11 and the subject to project the detection light L1 to different regions of the subject at different times, and then the photo detector 15 is able to receive the diffuse reflection light L2 from different regions of the subject. Then, the plurality of diffuse reflection light L2 received by the photo detector 15 can be further analyzed, and a more accurate diffuse reflection light L2 can be found from it, such as the diffuse reflection light L2 with the strongest intensity or the diffuse reflection light L2 with the strongest detected blood vessel pulsation, to improve the accuracy of the detection result.

In one embodiment of the invention, the movable device 17 may be connected to a driving unit 19, and the driving unit 19 is capable of driving the light emitting unit 13 and/or the photo detector 15 to displace relative to the carrier board 11 and/or the subject. For example, the slider 173 of the movable device 17 can be connected to a motor or a cylinder, and the motor or cylinder drives the slider 173 to displace along the slide rail 171, so that the light emitting unit 13 and/or the photo detector 15 move along the slide rail 171.

The light emitting unit 13 and the photo detector 15 may be connected to a power supply unit 18, wherein the power supply unit 18 is used to provide driving power to the light emitting unit 13 and the photo detector 15, so that the light emitting unit 13 can generate the detection light L1, and the photo detector 15 can be used to sense the diffuse reflection light L2. For example, the power supply unit 18 may supply power to the light emitting unit 13 and the photo detector 15 along the slide rail 171 of the movable device 17.

As shown in FIG. 2, the slide rail 171 of the movable device 17 may be a linear rail set along a first direction X, and the slider 173, the light emitting unit 13 and the photo detector 15 can be displaced relative to the slide rail 171 and the carrier board 11 along the first direction X.

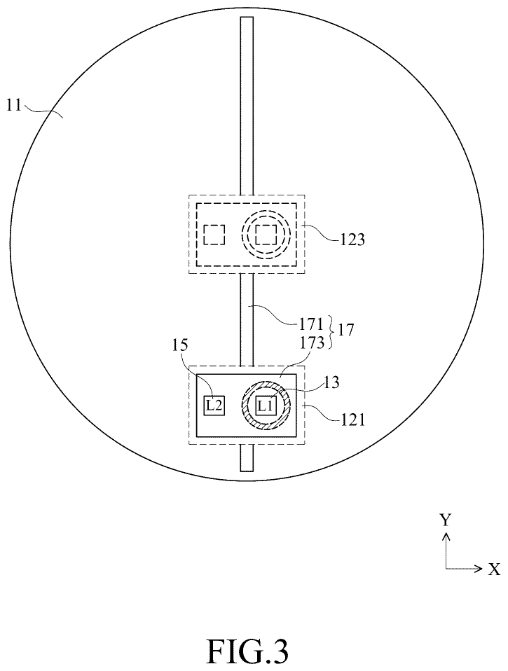

In another embodiment of the invention, as shown in FIG. 3, the slide rail 171 of the movable device 17 may be a linear rail set along a second direction Y, wherein the second direction Y is perpendicular to the first direction X. For example, the first direction X and the second direction Y are two mutually perpendicular directions parallel to the surface of the carrier board 11. The slider 173, the light emitting unit 13, and the photo detector 15 can be displaced relative to the slide rail 171 and the carrier board 11 along the second direction Y.

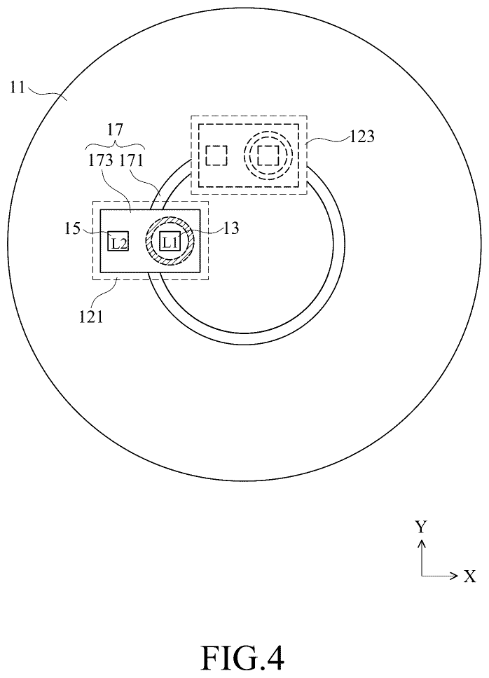

In another embodiment of the invention, as shown in FIG. 4, the slide rail 171 of the movable device 17 may be an arcuate, an annular, or a partially annular rail set on the surface of the carrier board 11. The light emitting unit 13 and the photo detector 15 are disposed on the same slider 173, and are connected to the slide rail 171 through the slider 173. In practical applications, the light emitting unit 13 and the photo detector 15 can be displaced along the slide rail 171 to project the detection light L1 onto different regions of the subject. During the displacement, the distance between the light emitting unit 13 and the photo detector 15 will remain constant.

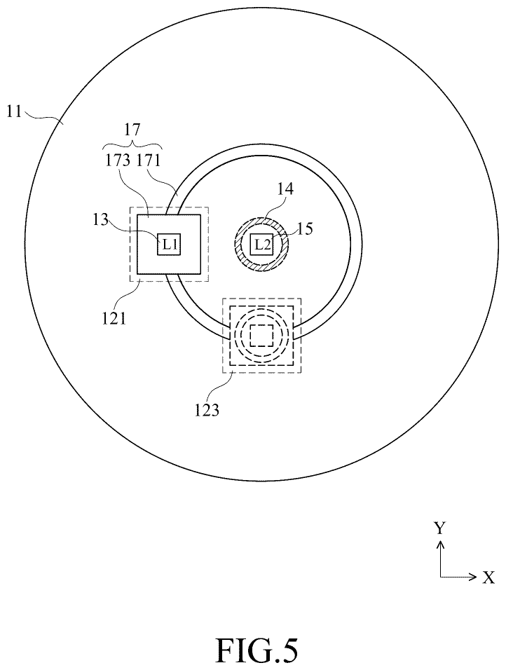

In another embodiment of the invention, as shown in FIG. 5, the slide rail 171 of the movable device 17 may be an arcuate, an annular, or a partially annular rail set on the surface of the carrier board 11, wherein the light emitting unit 13 is disposed on the slider 173, and the photo detector 15 is disposed at a fixed position on the carrier board 11. In practical applications, the light emitting unit 13 can be displaced along the slide rail 171 and project the detection light L1 to different regions of the subject. During the displacement, the distance between the light emitting unit 13 and the photo detector 15 will remain constant. For example, the slide rail 171 may be an annular rail, wherein the light emitting unit 13 is connected to the slide rail 171 through the slider 173, and the photo detector 15 is disposed on the carrier board 11 and located at the center position of the annular slide rail 171. In alternative embodiments, the photo detector 15 may be disposed on the slider 173, and the light emitting unit 13 may be disposed at the center position of the annular slide rail 171.

In the drawings of the above embodiments of the invention, the light emitting unit 13 and/or the photo detector 15 are capable of translational movement along the slide rail 171 to the first position 121 and the second position 123, and perform detection and/or measurement on the subject. In practical implementations, the number of positions at which the light emitting unit 13 and/or the photo detector 15 move along the slide rail 171 and perform detection may exceed two. For instance, the slider 173 may actuate the displacement of the light emitting unit 13 and/or the photo detector 15 along the slide rail 171 to arbitrary positions for measurement, thereby enabling the identification of a more accurate diffuse reflection light L2, such as the diffuse reflection light L2 exhibiting the highest intensity or the strongest blood vessel pulsation, to enhance the precision of the detection results.

In a further embodiment of the invention, the optical sensing module 10 may incorporate a barrier wall 14. This barrier wall 14, constructed from an opaque material, serves to physically separate the light emitting unit 13 and the photo detector 15, thereby preventing direct projection of the light (e.g., detection light L1) generated by the light emitting unit 13 onto the photo detector 15. As illustrated in FIG. 2, FIG. 3, and FIG. 4, the barrier wall 14 may be positioned on the slider 173, encasing the light emitting unit 13 and/or the photo detector 15 to preclude direct illumination of the photo detector 15 by the detection light L1 emitted from the light emitting unit 13. Alternatively, as depicted in FIG. 5, the barrier wall 14 may be situated on the carrier board 11, surrounding the photo detector 15.

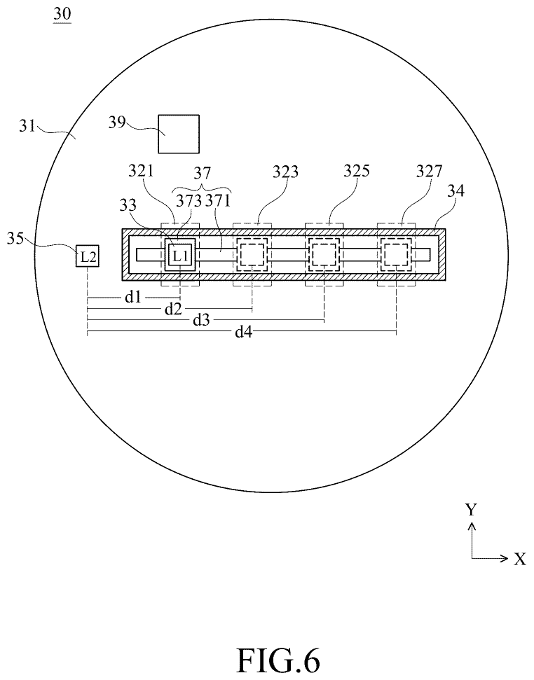

FIG. 6 is a schematic diagram of the optical sensing module according to another embodiment of the invention. The optical sensing module 30 of the invention can be disposed in a wearable device 20, and includes at least one carrier board 31, a light emitting unit 33, a photo detector 35, and a movable device 37. In one embodiment of the invention, the light emitting unit 33 can be disposed on the carrier board 31 through the movable device 37, while the photo detector 35 is affixed at a fixed location on the carrier board 31. In another embodiment of the invention, the photo detector 35 is disposed on the carrier board 31 through the movable device 37, while the light emitting unit 33 affixed at a fixed location on the carrier board 31. In this way, the light emitting unit 33 or the photo detector 35 can be displaced relative to the carrier board 31 to change the distance between the light emitting unit 33 and the photo detector 35, and obtain diffuse reflection light L2 data at different distances.

In this embodiment of the invention, the photo detector 35 is disposed on the carrier board 31, and the light emitting unit 33 is disposed on the slider 373 of the movable device 37. The movable device 37 is able to drive the light emitting unit 33 to displace relative to the photo detector 35 on the carrier board 31, and change the distance between the light emitting unit 33 and the photo detector 35. Specifically, the light emitting unit 33 is connected to the slide rail 371 through the slider 373, and the slider 373 drives the light emitting unit 33 to displace along the slide rail 371 relative to the photo detector 35, so that the detection light L1 generated by the light emitting unit 33 can be projected to different regions of the subject.

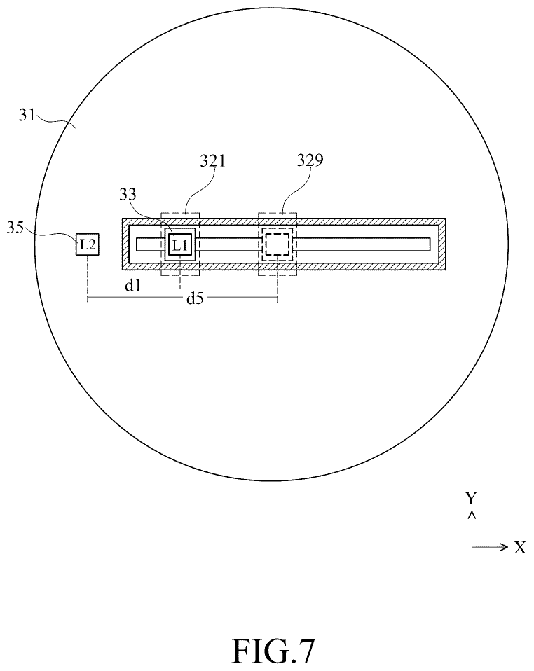

The movable device 37 can be connected to a driving unit 39, and the driving unit 39 is capable of driving the slider 373 and the light emitting unit 33 to displace along the slide rail 371 relative to the carrier board 31 and the photo detector 35. More specifically, in accordance with the physiological parameter types to be measured by the optical sensing module 30, the driving unit 39 can be employed to maneuver the light emitting unit 33 to a plurality of predefined positions, such as the first position 321, the second position 323, the third position 325, and/or the fourth position 327 as illustrated in FIG. 6, or the first position 321 and the fifth position 329 as depicted in FIG. 7.

In an embodiment of the invention, when the optical sensing module 30 is used to measure the blood oxygen of the subject, the driving unit 39 may drive the light emitting unit 33 to move to the first position 321, the second position 323, the third position 325 and the fourth position 327, wherein the photo detector 35 and the light emitting unit 33 of the first position 321, the second position 323, the third position 325 and/or the fourth position 327 respectively have a first distance d1, a second distance d2, a third distance d3 and a fourth distance d4.

The first distance d1, the second distance d2, the third distance d3, and the fourth distance d4 are different, wherein the fourth distance d4 is greater than the third distance d3, which is greater than the second distance d2, which is greater than the first distance d1. For example, the first distance d1 may be 6 mm, the second distance d2 may be 8 mm, the third distance d3 may be 10 mm, and the fourth distance d4 may be 14 mm. The absorption and scattering coefficients, used to derive the subject's blood oxygen levels, are determined by analyzing the ratios of diffuse reflection light L2 at these varying distances (d1, d2, d3, d4).

In another embodiment of the invention, when the optical sensing module 30 is used to measure the blood glucose of the subject, the driving unit 39 can drive the light emitting unit 33 to move to the first position 321 and the fifth position 329, wherein the photo detector 35 and the first position 321 and the fifth position 329 respectively have a first distance d1 and a fifth distance d5. The fifth distance d5 is greater than the first distance d1. For example, the fifth distance d5 may be 1.5 to 2.5 times the first distance d1. The optical sensing module 30, in conjunction with appropriate computational devices and methods, enables the derivation of the subject's blood glucose concentration.

The first distance d1, the second distance d2, the third distance d3, and the fourth distance d4 mentioned in the above embodiments of the invention may be the distance between the center position of the photo detector 35 and the center position of the light emitting unit 33. In other embodiments, the first distance d1, the second distance d2, the third distance d3, and the fourth distance d4 may be the distance between the edge position of the photo detector 35 and the edge position of the light emitting unit 33. Since the above first position 321, second position 323, third position 325, fourth position 327, and fifth position 329 are different, the diffuse reflection light L2 sensed by the photo detector 35 will be different. In practical applications, the position of the light emitting unit 33 can be adjusted by the driving unit 39 according to the measurement method of the optical sensing module 30 and the measured physiological parameters.

The above-mentioned driving unit 39 driving the light emitting unit 33 to move to the first position 321, the second position 323, the third position 325, the fourth position 327, and the fifth position 329 is merely one embodiment of the invention, and is not a limitation of the scope of the invention. In practical applications, the position of the light emitting unit 33 can be adjusted along the slide rail 371 according to the measurement method, the type of physiological parameters to be measured, and/or the intensity of the diffuse reflection light L2 received by the photo detector 35, and is conducive to improving the accuracy of the measurement.

As shown in FIG. 6, the slide rail 371 of the movable device 37 may be a linear rail set along a first direction X, and the slider 373 and the light emitting unit 33 can be displaced relative to the slide rail 371 and the carrier board 31 along the first direction X.

In another embodiment of the invention, the optical sensing module 30 may include a barrier wall 34, wherein the barrier wall 34 may be disposed on the carrier board 31. For example, the barrier wall 34 may be made of an opaque material and located between the light emitting unit 33 and the photo detector 35. The barrier wall 34 can be disposed between the light emitting unit 33 and the movable device 37 to prevent the light generating by the light emitting unit 33 from being directly projected onto the photo detector 35.

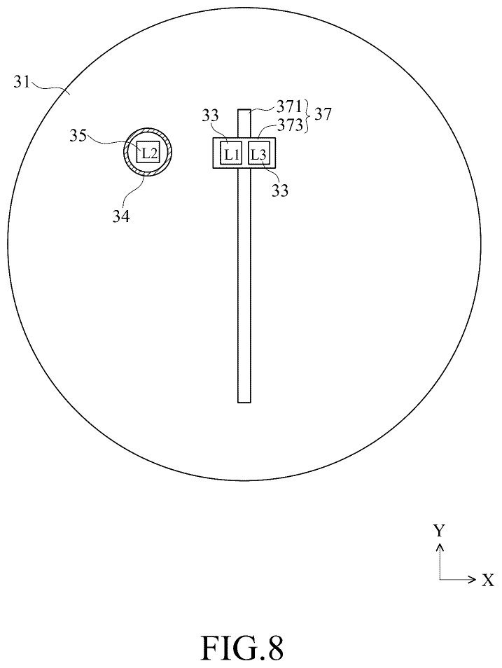

In another embodiment of the invention, as shown in FIG. 8, the slide rail 371 of the movable device 37 may be a linear rail set along a second direction Y, wherein the second direction Y is perpendicular to the first direction X. For example, the first direction X and the second direction Y are two mutually perpendicular directions parallel to the surface of the carrier board 31. The slider 373 and the light emitting unit 33 can be displaced relative to the slide rail 371 and the carrier board 31 along the second direction Y. In addition, a plurality of light emitting units 33 can be disposed on the slider 373 of the movable device 37. For example, each light emitting unit 33 on the slider 373 can generate detection light L1 and detection light L3 with different wavelength distributions or different light intensities, respectively.

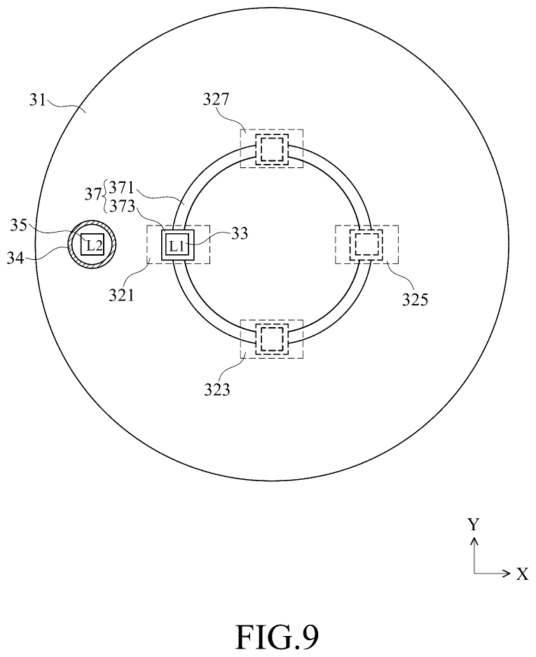

In another embodiment of the invention, as shown in FIG. 9, the slide rail 371 of the movable device 37 may be an arcuate, an annular, or a partially annular rail set on the surface of the carrier board 31, wherein the light emitting unit 33 is disposed on the slider 373, and the photo detector 35 is disposed on the carrier board 31. In practical implementation, the light emitting unit 33 can be translated along the slide rail 371, and thus the detection light L1 is able to project onto varying the area of the subject. During this translational movement, the distance between the light emitting unit 33 and the photo detector 35 changes dynamically. The barrier wall 34 is positioned to encircle the periphery of the photo detector 35.

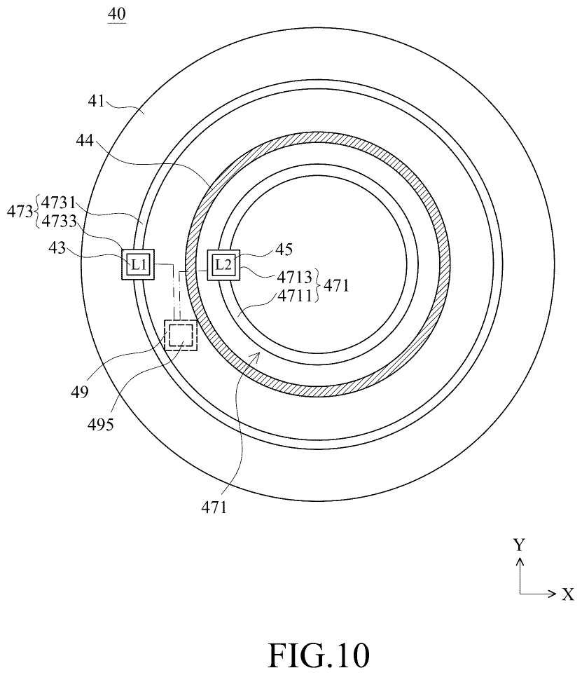

FIG. 10 is a schematic diagram of the optical sensing module according to another embodiment of the invention. The optical sensing module 40 of the invention can be disposed in a wearable device 20, and includes at least one carrier board 41, a light emitting unit 43, a photo detector 45, a first movable device 471 and a second movable device 473, wherein the photo detector 45 is disposed on the first movable device 471, and the light emitting unit 43 is disposed on the second movable device 473.

The structures of the first movable device 471 and the second movable device 473 are similar to the movable devices 17/37 of the previous embodiments, and are disposed on the carrier board 41. For example, the first movable device 471 includes a first slide rail 4711 and a first slider 4713, and the second movable device 473 includes a second slide rail 4731 and a second slider 4733. The first movable device 471 and the second movable device 473 are respectively used to drive the photo detector 45 and the light emitting unit 43 to displace relative to the carrier board 41, and change the distance between the light emitting unit 43 and the photo detector 45. For instance, the first slider 4713 can facilitate the displacement of the photo detector 45 relative to the carrier board 41, enabling the photo detector 45 to receive diffuse reflection light L2 from diverse locations on the subject. This arrangement enables the detection of diffuse reflection light L2 from varying tissue depths within the subject, enhancing measurement accuracy and comprehensiveness.

In the drawings of the invention, the first movable device 471 and the second movable device 473 are annular and have different sizes. For example, the first movable device 471 may be located inside the second movable device 473. In alternative embodiments, the first movable device 471 may be located outside the second movable device 473, wherein the second slide rail 4731 surrounds the periphery of the first slide rail 4711. For example, the first movable device 471 and the second movable device 473 include, but are not limited to, a concentric circle structure, a concentric ellipse structure, or a concentric ring structure. In addition, an annular barrier wall 44 can be disposed between the first movable device 471 and the second movable device 473. The annular shape of the first movable device 471 and the second movable device 473 is only one embodiment of the invention, and is not a limitation of the scope of the invention. In other embodiments, the first movable device 471 and the second movable device 473 may be other geometric shapes, such as a straight line, an arcuate, a partially annular, or one of a combination of the above geometric shapes.

In another embodiment of the invention, the second slide rail 4731 encircles the periphery of the first slide rail 4711, or conversely, the first slide rail 4711 encircles the periphery of the second slide rail 4731, thereby forming a spherical scanning configuration. This allows the light emitting unit 43 and the photo detector 45 to undergo relative displacement within a three-dimensional space, enabling omnidirectional scanning or detection of a subject.

In another embodiment of the invention, at least one driving unit 49 can be respectively connected to the first movable device 471 and the second movable device 473, and configured to actuate the first slider 4713 and the second slider 4733 to undergo relative movement either synchronously or asynchronously, thereby enabling omnidirectional spatial scanning of a subject.

In another embodiment of the invention, the driving unit 49 includes a microprocessor 495, which can control the position and/or displacement speed of the first slider 4713 and the second slider 4733, thereby realizing intelligent scanning detection of a subject.

In the embodiments of FIG. 6, FIG. 7, FIG. 8 and FIG. 9 of the invention, the light emitting unit 33 is disposed on the movable device 37, and the photo detector 35 is disposed on the carrier board 31. In other embodiments, the photo detector 35 can be disposed on the movable device 37, and the light emitting unit 33 can be disposed on the carrier board 31. Similarly, the movable device 37 can be used to drive the photo detector 35 to displace relative to the carrier board 31 and/or the light emitting unit 33 to change the distance between the light emitting unit 33 and the photo detector 35.

For the convenience of explanation, in the embodiments of FIG. 2, FIG. 3, FIG. 4, FIG. 5, FIG. 6, FIG. 7, FIG. 9 and FIG. 10 of the invention, only one light emitting unit 13/33/43 and one photo detector 15/35/45 are drawn in the embodiments, and the description is made with a single light emitting unit 13/33/43 and a single photo detector 15/35/45. In other embodiments, the number of light emitting units 13/33/43 and photo detectors 15/35/45 can be plural. For example, multiple light emitting units 13/33/43 and/or multiple photo detectors 15/35/45 can be disposed on the carrier board 11/31/41 or the slider 173/373.

When the optical sensing module Oct. 30, 1940 has multiple light emitting units 13/33/43, each light emitting unit 13/33/43 can be used to generate detection light L1, L3 with different wavelength distributions or different light intensities, respectively. By way of example, the light emitting unit 13/33/43 may comprise, but is not limited to, a light emitting diode, and the detection light L1 may be a steady-state light with a wavelength within the range of 600 to 1100nm.

In addition, in other embodiments of the invention, the light source of the light emitting unit 13/33/43 may comprise a plurality of micro light emitting diodes (Micro LEDs), wherein each individual micro light emitting diode is capable of generating detection light L1 with distinct wavelength distributions.

In an alternative embodiment of the invention, the light emitting unit 13/33/43 may be a single light emitting diode, and the power supply unit 18 may be configured to supply varying magnitudes of driving current to the light emitting unit 13/33/43. This enables the light emitting unit 13/33/43 to generate detection light L1, L3 with distinct wavelength distributions or varying light intensities.

In one embodiment of the invention, the driving unit 19/39 may be an electric device such as a stepping motor, or it may be a mechanical driving device that is not driven by electricity, such as, but not limited to, a gear and lever system, a gravity-driven system, etc. The driving unit 19/39 can not only drive the slider 173 to move to a plurality of specific positions on the slide rail 171, but also control and drive the slider 173 to move at different speeds.

In other embodiments of the invention, the quantity of driving units 19/39 may be singular. For instance, a solitary driving unit 19/39 may be employed to concurrently or sequentially regulate the displacement of the first slider 4713 and/or the second slider 4733 to distinct positions. Alternatively, two independent driving units 19/39 may be utilized, with each connected to the first movable device 471 or the second movable device 473, thereby enabling individual actuation of the first slider 4713 or the second slider 4733 as per design specifications.

As shown in FIG. 1, the wearable device 20 is a smart watch, and the optical sensing module 10 (30/40) is disposed on the watch body 21 of the watch and used to project the detection light L1 onto the wrist of the subject. The wearable device 20 may include at least one processor, at least one memory or at least one wireless transmission chip, such as a Bluetooth chip, wherein the processor, memory or wireless transmission chip can be disposed on the carrier board 11/31/41.

The processor within the wearable device is capable of transforming the diffuse reflection light L2 signal, as received by the optical sensing module Oct. 30, 1940, into optical parameters, such as the absorption coefficient and scattering coefficient. Subsequently, calculations are performed on these optical parameters to derive the subject's physiological parameters, including but not limited to blood glucose concentration and blood oxygen saturation. The specific computational methodologies are well-documented in the relevant prior art and are readily understood and practiced by those with ordinary skill in the art of this invention. As these computational methods do not constitute the core technical features of the present invention, they will not be elaborated upon herein.

In one embodiment of the invention, the processor, memory, wireless transmission chip, optical sensing module Oct. 30, 1940, driving unit 19/39 and/or power supply unit 18 of the wearable device 20 may be integrated on the same carrier board and packaged together to form a Co-Package Optics (CPO) device.

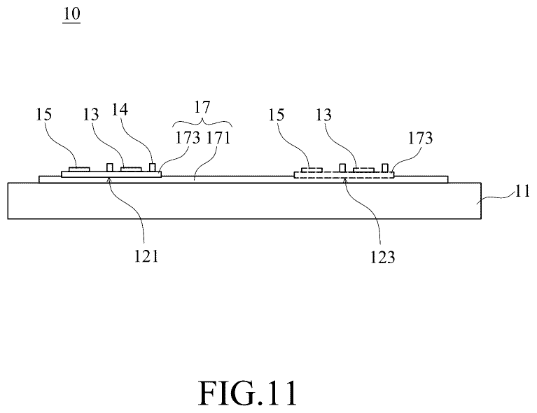

Referring to FIG. 11, in one embodiment of the invention, the carrier board 11 of the optical sensing module 10 is configured as a planar substrate. Please referring to FIG. 2 or FIG. 3, the slide rail 171 of the movable device 17 may be situated on a common horizontal plane or a common vertical plane. Consequently, the slider 173, the light emitting unit 13, the photo detector 15, the first position 121, and the second position 123 are all located on the same plane.

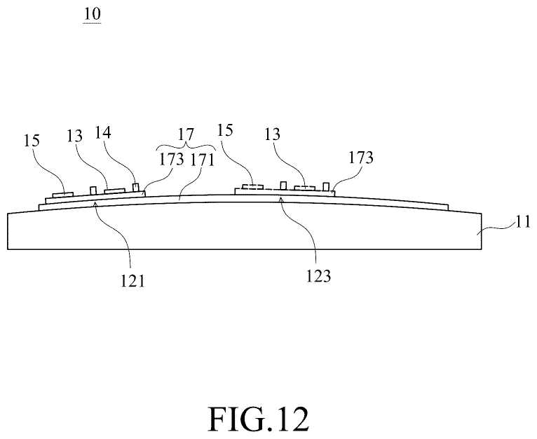

In a further embodiment of the invention, the carrier board 11 of the optical sensing module 10 is configured as a non-planar substrate, including but not limited to a concave, concave-convex, stepped, or convex form, as illustrated in FIG. 12. The slide rail 171 of the movable device 17 is capable of displacement along the surface of the non-planar carrier board 11. Consequently, the first position 121 and the second position 123 may be situated on distinct planes. This arrangement allows the light emitting unit 13 and the photo detector 15, positioned on the slider 173, to undergo relative movement in a three-dimensional space, thereby enabling three-dimensional displacement of the light emitting unit 13 and the photo detector 15 relative to a subject for comprehensive detection of non-planar or curved subjects.

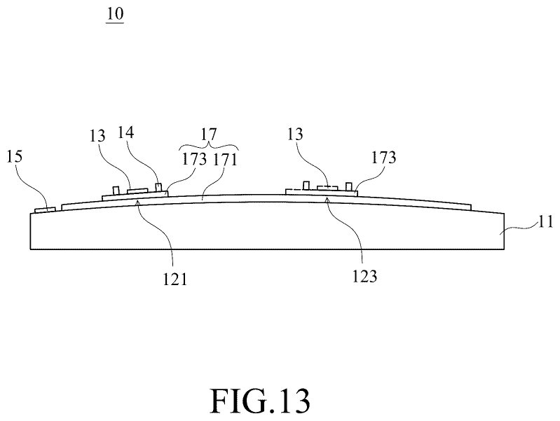

FIG. 13 is a side view of another embodiment of the invention. The carrier board 11 of the optical sensing module 10 is a non-planar carrier board, wherein the slide rail 171 of the movable device 17 can be disposed along the surface of the non-planar carrier board 11. The light emitting unit 13, mounted on the slider 173, is capable of translational movement along the non-planar slide rail 171 between the first position 121 and the second position 123, wherein the first position 121 and the second position 123 exhibit differing horizontal or vertical elevations. The photo detector 15 is affixed at a stationary location on the carrier board 11. In other embodiments, the photo detector 15 mounted on the slider 173 can move along the non-planar slide rail 171 at the first position 121 and the second position 123, and the light emitting unit 13 is affixed at a stationary location on the carrier board 11. This configuration facilitates omnidirectional detection of non-planar or curved subjects.

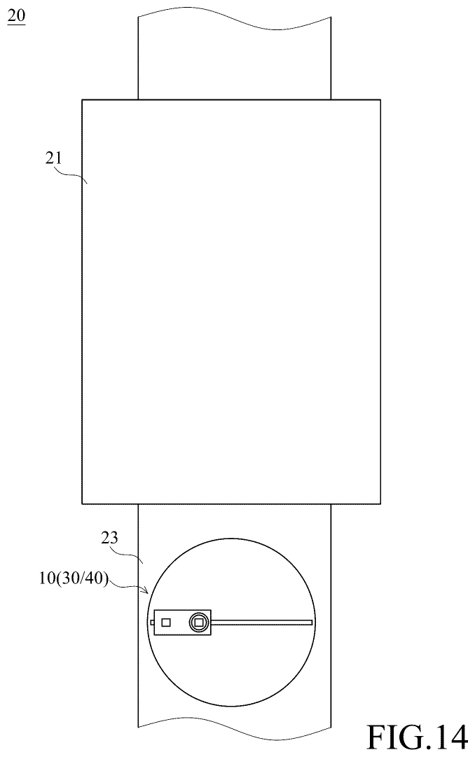

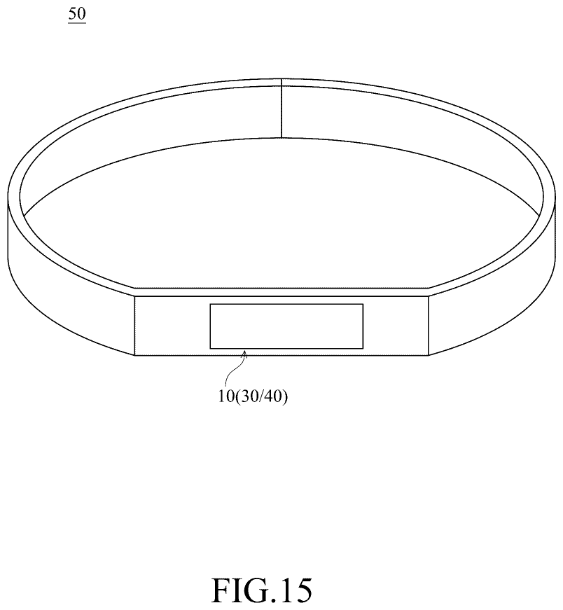

In practical applications, as shown in FIG. 14, the optical sensing module Oct. 30, 1940 may be disposed on the strap 23 of the smart watch, and can project the detection light L1 onto the wrist of the subject and receive the diffuse reflection light L2. As shown in FIG. 15, the optical sensing module Oct. 30, 1940 may be disposed on the wearable device 50, and can project the detection light L1 onto the neck of the subject. For example, the wearable device 50 can be a collar.

In one embodiment of the invention, the carrier board 11/31/41 may be connected to another movable device (not shown) to drive the carrier board 11/31/41, the light emitting unit 13/33/43 and the photo detector 15/35/45 to displace in a direction perpendicular to the first direction X and the second direction Y, so as to change the distance between the carrier board 11/31/41, the light emitting unit 13/33/43 and the photo detector 15/35/45 and the subject.

In another embodiment of the invention, the optical sensing module Oct. 30, 1940 may be fabricated utilizing a silicon photonics manufacturing process. For example, the processor, memory, wireless transmission chip, optical sensing module Oct. 30, 1940, driving unit 19/39, and/or power supply unit 18 of the wearable device 20/50 may be integrated onto a single wafer substrate, thereby forming a silicon photonics structure. The implementation of co-packaging and silicon photonics process technologies facilitates a reduction in the volume of the optical sensing module Oct. 30, 1940, a decrease in the power consumption of the wearable device 20/50, and an enhancement of the transmission speed.

The foregoing descriptions are merely preferred embodiments of this disclosure, and are not intended to limit the scope of this disclosure, that is, all equivalent changes and modifications made according to shapes, structures, features and spirits described in the scope of the claims of this disclosure shall fall within the scope of the claims of this disclosure.

What is claimed is:

1. An optical sensing module, comprising:
a carrier board;
at least one light emitting unit for generating a detection light, wherein the detection light is configured to project onto a subject to generate a diffuse reflection light;
at least one movable device disposed on the carrier board, and including a slide rail and a slider, wherein the slider is displaceable along the slide rail, and the light emitting unit is disposed on the slider, the movable device is used to drive the light emitting unit to displace relative to the carrier board to project the detection light onto varying regions of the subject; and
at least one photo detector for receiving the diffuse reflection light.

2. The optical sensing module of claim 1, wherein the photo detector is disposed on the slider of the movable device, a consistent distance is maintained between the light emitting unit and the photo detector, and the movable device is configured to drive the light emitting unit and the photo detector to displace relative to the carrier board.

3. The optical sensing module of claim 1, wherein the photo detector is disposed on the carrier board, and the movable device is configured to drive the light emitting unit to displace relative to the photo detector on the carrier board, to change a distance between the light emitting unit and the photo detector.

4. The optical sensing module of claim 1, wherein the slide rail is a straight line, an arcuate, an annular or a partially annular.

5. The optical sensing module of claim 1, wherein the light emitting unit includes a plurality of light sources for respectively generate the detection light with a distinct wavelength.

6. The optical sensing module of claim 1, wherein the slider is connected to a driving unit, and the driving unit is configured to actuate displacement of the slider to a plurality of positions along the slide rail.

7. The optical sensing module of claim 1, further comprising a barrier wall disposed between the light emitting unit and the photo detector, to block the detection light generated by the light emitting unit from directly projecting onto the photo detector.

8. The optical sensing module of claim 1, wherein the carrier board is a planar carrier board or a non-planar carrier board.

9. An optical sensing module, comprising:
a carrier board;
at least one light emitting unit for generating a detection light, wherein the detection light is configured to project onto a subject to generate a diffuse reflection light;
at least one photo detector for receiving the diffuse reflection light; and
a first movable device disposed on the carrier board, including a first slide rail and a first slider, the first slider displacing along the first slide rail, and the photo detector disposed on the first slider, wherein the first slider is configured to drive the photo detector to displace relative to the carrier board, and the photo detector is able to receive the diffuse reflection light at a plurality of positions.

10. The light sensing module of claim 9, wherein the light emitting unit is disposed on the carrier board, and the first slider is configured to drive the photo detector to displace relative to the light emitting unit, so as to change a distance between the light emitting unit and the photo detector.

11. The optical sensing module as claimed in claim 9, wherein the slide rail is a straight line, an arcuate, an annular or a partially annular.

12. The optical sensing module of claim 9, further comprising a second movable device disposed on the carrier board, wherein the second movable device includes a second slide rail and a second slider, the second slider is displaceable along the second slide rail, wherein the light emitting unit is disposed on the second slider, and the second slider is configured to drive the light emitting unit to displace relative to the carrier board.

13. The optical sensing module of claim 12, further comprising at least one driving unit connected to the first movable device and the second movable device, and configured to drive the first slider to displace relative to the first slide rail, and configured to drive the second slider to displace relative to the second slide rail.

14. The optical sensing module of claim 13, wherein the driving unit is used to control the first slider and the second slider to perform synchronous or asynchronous displacement.

15. The optical sensing module of claim 13, wherein the driving unit includes a microprocessor, configured to control the position or a displacement speed of the first slider and the second slider.

16. The optical sensing module of claim 9, further comprising a barrier wall disposed between the light emitting unit and the photo detector, to block the detection light generated by the light emitting unit from directly projecting onto the photo detector.

17. The optical sensing module of claim 12, wherein the second sliding rail surrounds the periphery of the first sliding rail.

18. The optical sensing module of claim 9, wherein the carrier board is a planar carrier board or a non-planar carrier board.
